(19) United States
(12) Patent Application Publication
(10) Pub. No.: US 2024/0081672 A1
(43) Pub. Date: Mar. 14, 2024

(54) MEASURING DEVICE AND MEASURING METHOD TO MEASURE WRIST OR ANKLE MERIDIAN IMPEDANCE

(71) Applicant: Wellness Allied Inc, Natick, MA (US)

(72) Inventors: Weihui Li, Natick, MA (US); Tao ZHANG, Natick, MA (US)

(21) Appl. No.: 17/944,261

(22) Filed: Sep. 14, 2022

(51) Int. Cl. A61B 5/0531; A61B 5/00

(52) U.S. Cl. CPC A61B 5/0531; A61B 5/4854; A61B 5/681

(57) ABSTRACT

A meridian impedance measuring device that can be worn on a wrist or an ankle is provided, including: one or more stretchable parts, constituting part of a watchband, to allow a length of the watchband to be stretched by 1.3 times or more than 1.3 times of an original length; an electrode holding part, including six or more individual pieces that are connected with the stretchable parts, wherein, each piece may hold a pair of electrodes; six or more pairs of electrodes, wherein, one of each pair of the electrodes is a measuring electrode, and the other is a reference electrode, and the six or more pairs of electrodes are arranged on the electrode holding part around the wrist or the ankle; an integrated circuit part for measuring skin impedance; and a case, to house the integrated circuit part.

### TECHNICAL FIELD

The present disclosure generally relates to a meridian impedance measurement technology in traditional Chinese medicine, and more particularly, to a wearable device onto a wrist or an ankle to measure skin impedance.

### BACKGROUND

Many scientists or clinicians have conducted a large amount of skin impedance measurement studies and proposed some methods and theories to evaluate human health.

The patent application US20210369134A1 proposes a measurement method for simultaneously measuring 12 meridians. However, there is currently no design on the market that can easily and accurately measure meridian skin impedance. Main difficulties here are: 1) how to fix electrodes on a wristband/watch/anklet; 2) how to distribute the electrodes evenly around a wrist/ankle; 3) how to put the wristband/watch/anklet conveniently on wrists/ankles of different sizes; 4) how to ensure a skin impedance value of each meridian to be measured relatively accurately; and 5) how to place a plurality of wires that connect electrodes to integrated circuits.

### SUMMARY OF THE DISCLOSURE

In view of the above-described problems in the prior art, the present disclosure is proposed.

According to one aspect of the present disclosure, a measuring device to measure skin impedance, which is worn onto a wrist or an ankle, comprising: one or more stretchable parts, which constitute part of a watchband and allow the length of the watchband to be stretched by 1.3 times or more than 1.3 times of an original length; an electrode holding part, including six or more individual pieces that are connected with the stretchable parts, wherein, each piece may hold a pair of electrodes; six or more pairs of electrodes, wherein, one of each pair of the electrodes is a measuring electrode, and the other is a reference electrode, and the six or more pairs of electrodes are arranged on the electrode holding part around the wrist or the ankle; an integrated circuit part for measuring skin impedance; and a case, to house the integrated circuit part.

Optionally, each electrode is made of two pieces of metal sleeved together; the two pieces of metal partially overlap with each other, with a spring connected within to allow the two pieces of metal to move relative to each other; one piece of metal is in contact with the skin, and the other piece of metal is connected to the integrated circuit through a wire.

Optionally, all reference electrodes are connected to a common wire, and then connect to the integrated circuit; and each measuring electrode is connected to the integrated circuit through an individual wire.

Optionally, the device is worn onto a wrist or an ankle by passing through a hand or a foot with the stretchable parts being stretched or compressed.

Optionally, each piece of the electrode holding part has two protruding hollow cylinders along a longitudinal direction of the wrist or the ankle.

Optionally, in the hollow portion of the cylinder, a diameter on an end close to the skin is greater than a diameter on the other end away from the skin.

Optionally, the electrode is inserted into the hollow cylinder from the bottom of the electrode holding piece; one end of the electrode is in contact with the skin, and the other end of the electrode is mounted onto the electrode holding piece and is connected to the integrated circuit through a wire.

Optionally, the stretchable part is made of silicone, rubber, or plastic.

Optionally, the stretchable part is punched with pairs of holes along a circumferential direction of the wrist or ankle.

Optionally, a zigzag groove is cut on the stretchable part to house the wires that connect the electrodes and the integrated circuit.

Optionally, each pair of holes on the stretchable part are set on the two cylinders of each electrode holding piece.

Optionally, each piece of the electrode holding part and the connected stretchable part are covered with a plate, to hide the stretchable part, electrodes, and wires.

Optionally, the adjacent plates are partly overlapped; when the watchband is stretched, the overlapped parts are extended to cover the stretchable part and the wires.

Optionally, the stretchable parts are springs.

Optionally, a sliding rod is fixed on each electrode holding piece along a circumferential direction of the wrist or the ankle; the spring is put on the sliding rod.

Optionally, the sliding rod is further connected with one end of a cover, next to the spring, and the other end of the cover is connected to an adjacent electrode holding piece; when two electrode holding pieces are pulled away from each other, the end of the cover that is next to the spring compresses the spring along the sliding rod.

Optionally, the wires that connect electrodes to the integrated circuits, the springs, the cylinders, the sliding rods are hidden by covers.

Optionally, the adjacent covers are partly overlapped; when the watchband is stretched, the overlapped parts are extended to cover the wires, springs, cylinders and sliding rods.

The above-described embodiments of the present disclosure solve the problems of measuring skin impedances on multiple meridians, and have at least the following advantages: 1) the electrodes are distributed evenly around the wrist/ankle; 2) a few different sizes of watchbands may be conveniently produced to fit all different sizes of wrists/ankles; 3) the design of a spring within the electrode allows the electrode to contact the irregular wrist/ankle reliably; 4) the design of the watchband allows the wires to be hidden inside the watchband securely; and 5) the curved, redundant, or stretchable wire design allows the watchband to be stretched.

### BRIEF DESCRIPTION OF THE DRAWINGS

FIG. 1 shows an exemplary schematic diagram of a wristwatch for measuring skin impedance around a wrist according to the prior patent application WO2021/247449A1.

FIG. 2A shows a common design of an openable watchband which may be buckled on a wrist. FIG. 2B shows a schematic diagram of the relative sizes of a hand and a wrist. When the watch according to the embodiments of the present disclosure is worn on the wrist through the relatively big hand, the watchband needs to be stretched by more than 1.3 times.

FIG. 3 shows a front view of the skin-contacting side of the watch case and the stretchable part of the watch according to the embodiment of the present disclosure,

FIG. 4 shows an exemplary schematic diagram of the other side of the watch case and the stretchable part of the watch shown in FIG. 3 according to an embodiment of the present disclosure.

FIG. 5A to FIG. 5C show exemplary schematic diagrams of the electrode holding part according to an embodiment of the present disclosure.

FIG. 6 shows an effect diagram after setting the stretchable part shown in FIG. 4 on the electrode holding part shown in FIG. 5C.

FIG. 7A to FIG. 7D show exemplary schematic diagrams of an electrode design according to an embodiment of the present disclosure.

FIG. 8 shows an exemplary schematic diagram of a structure after mounting the electrodes onto the electrode holding part shown in FIG. 6 and after wiring the electrodes.

FIG. 9 shows an exemplary schematic diagram of a skin-contacting side of the watch shown in FIG. 8.

FIG. 10 shows an exemplary schematic diagram of a plate that covers the electrode holding piece according to an embodiment of the present disclosure.

FIG. 11 shows an exemplary schematic diagram of the watch after the plates cover all the electrode holding pieces on the watchband in FIG. 8 according to an embodiment of the present disclosure.

FIG. 12 shows an overall exemplary schematic diagram of another watch design according to an embodiment of the present disclosure.

FIG. 13 shows an exemplary schematic diagram of the watch shown in FIG. 12 after the watchband is stretched according to an embodiment of the present disclosure.

FIG. 14 shows an exemplary schematic diagram of the watch shown in FIG. 13 when a cover 1280 and a plate 1320 are removed.

FIG. 15 shows an exemplary schematic diagram of the cover 1280 shown in FIG. 13.

FIG. 16 shows an exemplary schematic diagram of an internal structure of the electrode holding piece 1230 shown in FIG. 12.

FIG. 17 shows a schematic diagram of the watch when the electrode holding piece and the adjacent cover are pulled away from each other.

### DETAILED DESCRIPTION

The technical solutions of the present disclosure will be further described below in conjunction with the drawings and through specific implementations.

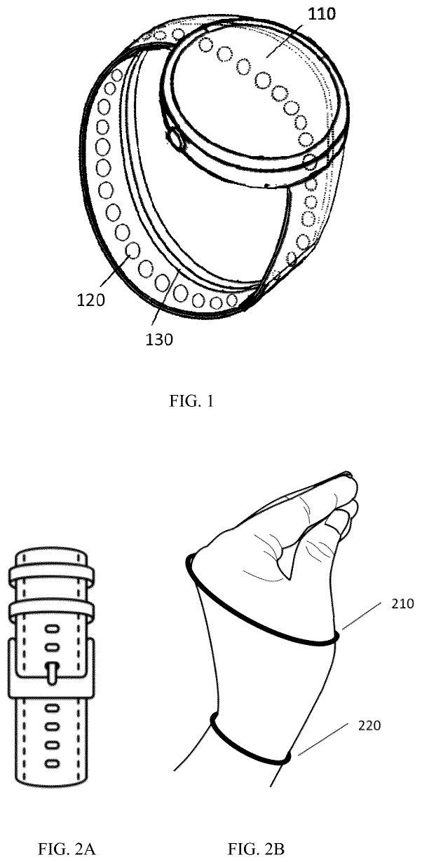

FIG. 1 shows a schematic diagram of a prior patent application WO2021/247449A1. The diagram shows a watch for measuring skin impedance around a wrist, thereby deriving impedance values of six meridians on the wrist.

FIG. 2. 2A shows a watchband that is openable. The user needs to buckle the two ends of the watchband when wearing the watch. In the design of a watch with plenty of electrodes, like the one shown FIG. 1, it is not practical to adopt an openable watchband.

Therefore, the present disclosure provides a solution of a watch with a stretchable watchband that can be worn onto the wrist through a hand. FIG. 2B shows the relative sizes of a hand and a wrist. 210 is the largest position when passing through the hand, and 220 is the size of the wrist. The circumference of 210 is usually 1.3 times more that of 220.

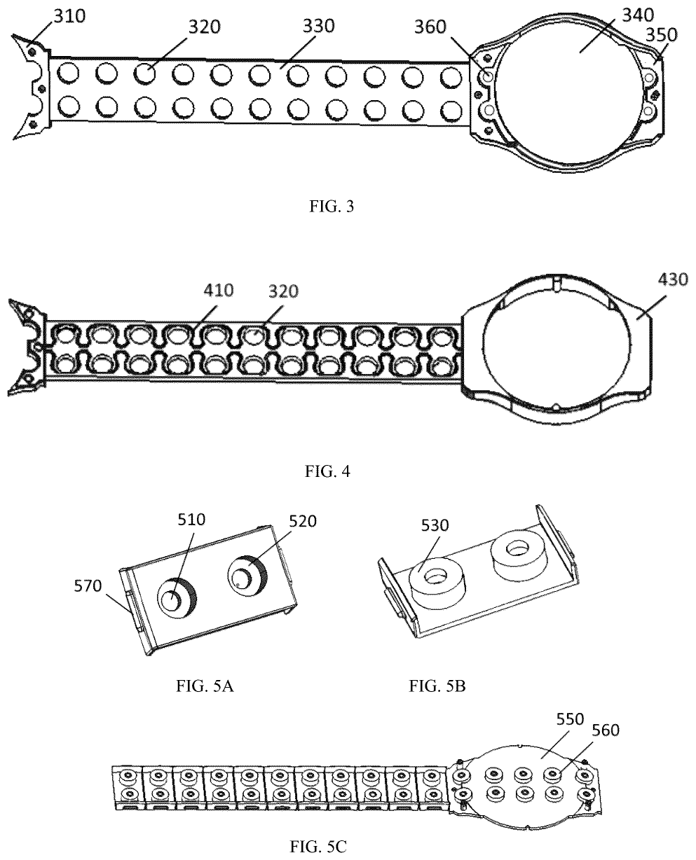

FIG. 3 to FIG. 11 show designs of a watch according to an embodiment of the present disclosure.

FIG. 3 shows a front view of a skin-contacting side of the stretchable part of a watch and a watch case, including: a skin-contacting side of a watch case 350, a watch case hollow portion 340, a watchband 330 made of a stretchable material and a connecting piece that may be inserted into the back side of the watch case 350 to form a closed watchband. The watch case hollow portion 340 is used to house an integrated circuit and a watch face, and the watchband 330 is punched with pairs of holes 320. 360 is an opening on the watch case for electrode. The stretchable material may be a silicone.

FIG. 4 shows the other side of the watch shown in FIG. 3. On this side, the stretchable part is cut out with two narrow grooves 410 to place wires, which are used to connect the electrodes to the integrated circuit. 430 is the front side of the watch case 350.

FIG. 5A to FIG. 5B show an electrode holding piece. FIG. 5A is a skin-contacting side; 510 and 520 are two holes of different diameters to mount an electrode. 570 is a protruding strip to connect with a cover to hide wires and electrodes. FIG. 5B is the other side of the electrode holding piece. 530 is a hollow cylinder with holes 510 and 520. The electrode shown in FIG. 7 is inserted into the holes 520 and 510 and screwed onto cylinder 530. FIG. 5C shows an array of electrode holding pieces and the watch case 550. 560 is a hole on the watch case to mount the electrode.

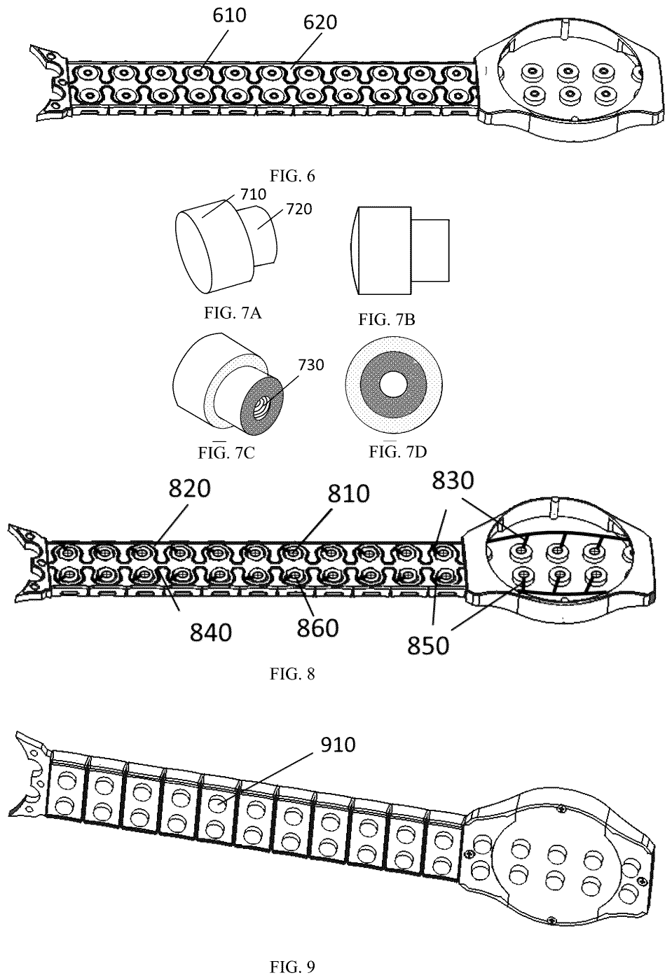

FIG. 6 shows a diagram after setting the stretchable part of the watchband shown in FIG. 4 onto the electrode holding part and the watch case shown in FIG. 5C. 620 is the stretchable part, corresponding to 330 in FIGS. 3; and 610 is the hollow cylinder on the electrode holding part to mount the electrodes.

FIG. 7A to FIG. 7D show an electrode design. In FIG. 7A, 710 and 720 are two pieces of metal that may move relative to each other; and the two pieces of metal 710 and 720 are connected by a spring. 710 is a metal piece contacting the skin, and 720 is used to connect with the electrode holding piece in FIG. 5. The hollow portion of 720 may be designed with threads 730 (FIG. 7C), to mount the electrode by a screw. FIG. 7B and FIG. 7D are side and front views of FIG. 7A, showing the relative diameters of the two cylindrical metal pieces. The electrode design with a spring within the electrode helps the electrodes to effectively contact the irregular shapes of wrists/ankles of most of the population by just providing a limited number of watchband sizes.

FIG. 8 shows a schematic diagram of a watchband and watch case after electrodes are mounted on and wires are connected to the watch shown in FIG. 6. Of each pair of electrodes, one is a measuring electrode 810 and the other is a reference electrode 860. All the reference electrodes are connected to a common reference electrode wire 840 and then connected to the integrated circuit in the watch case. The measuring electrodes are connected to the integrated circuit in the watch case through individual wires 830 that form a measuring electrode bus 820. The number of wires in the measuring electrode bus 820 equal the number of measuring electrode.

FIG. 9 shows a skin-contacting side of a watch after the electrodes are mounted. 910 is an electrode.

FIG. 10 is a plate 1010 covering an electrode holding piece to hide the wires, the screws, the cylinders, etc.

FIG. 11 shows a schematic diagram of a watchband and watch case after the plates has covered all the electrode holding pieces shown in FIG. 8.

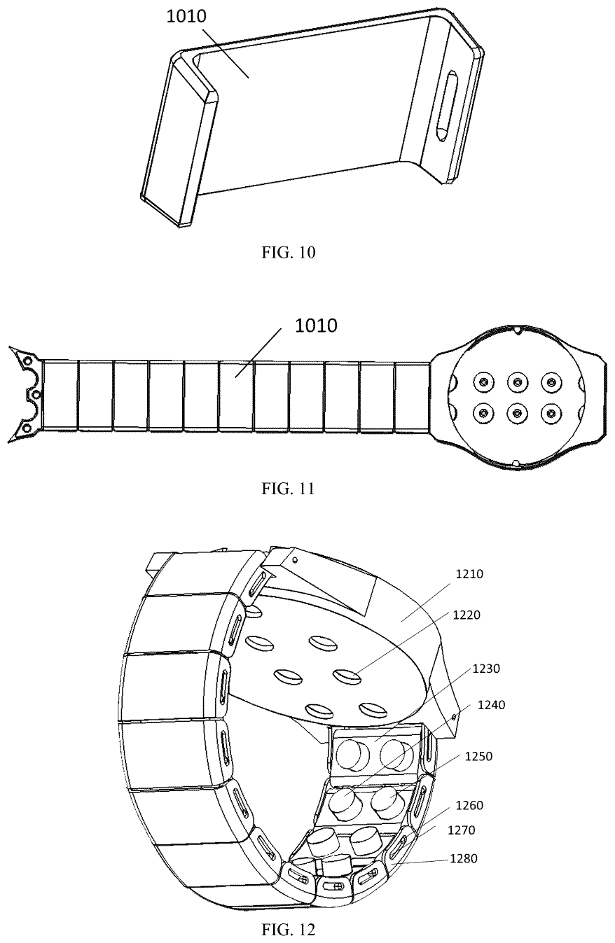

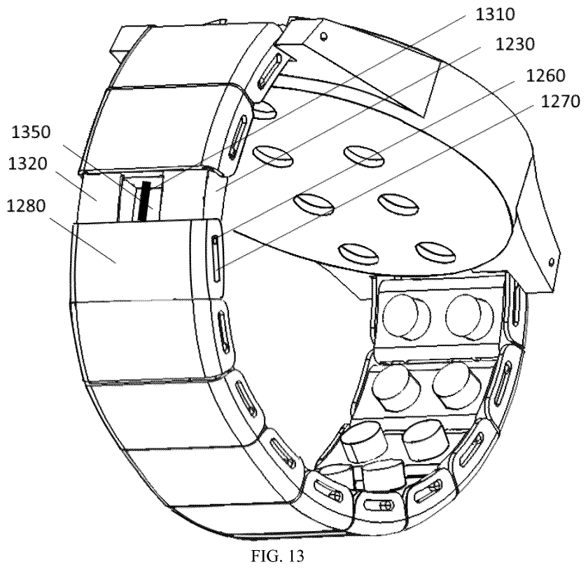

FIG. 12 to FIG. 17 demonstrate another design of a watch according to another embodiment of the present disclosure.

FIG. 12 shows an overall schematic diagram of the watch. 1210 is the watch case. The holes 1220 at the bottom of the watch case are for the electrodes. 1230 is the electrode holding piece. 1240 and 1250 are a measuring electrode and a reference electrode respectively. 1260 is a nodule on the electrode holding piece. It can slide in a narrow groove 1270 on the side of the cover 1280.

FIG. 13 shows a schematic diagram after the cover 1280 and the electrode holding piece 1230 are pulled away from each other, the nodule 1260 slides along the groove 1270. In the middle of the electrode holding piece 1260, there is a small compartment 1350. Inside 1350, there is a sliding rod 1310 along a circumferential direction of the wrist/ankle. There are also small plates 1320 to cover the electrodes, the wires, etc. when the watchband is stretched, and the interior components are exposed.

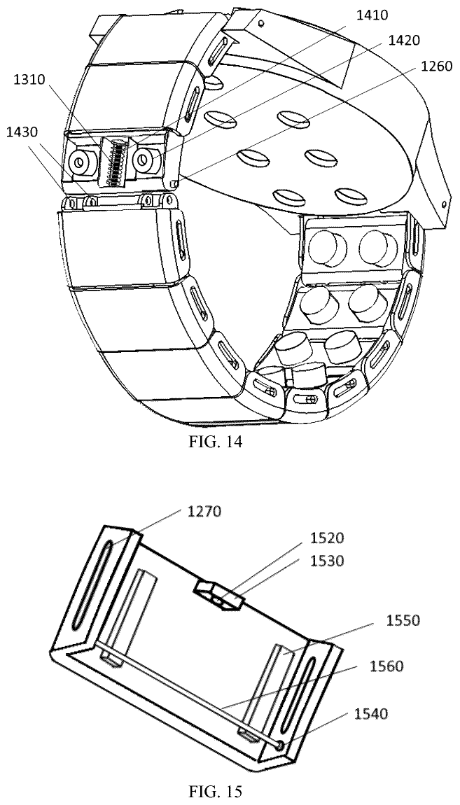

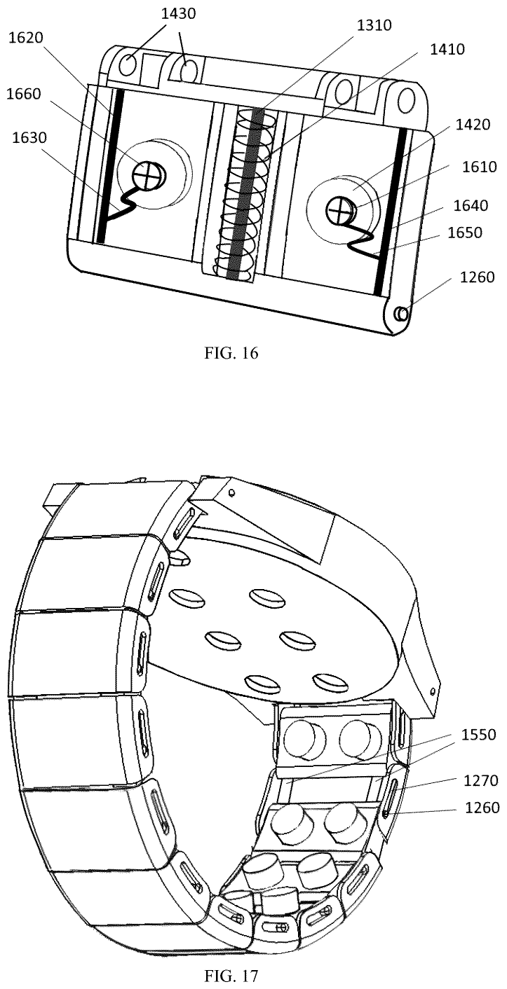

FIG. 14 shows a schematic diagram when the cover 1280, the plates 1320 in FIG. 13, the electrodes and the wires in FIG. 16 are removed. Bolt holes 1430 are designed to connect with one end of the cover 1289 through a rod (1560 in FIG. 15). The other end of the cover is connected on the sliding rod 1310 through a pressing piece (1530 in FIG. 15). A spring 1410 is put on the sliding rod between the two ends of the cover. The spring is compressed by the pressing piece when the watchband is stretched and is hidden under the cover. The hollow cylinder 1420 is used to mount the electrodes.

FIG. 15 shows an inner side of the cover 1280 in FIG. 13. The thin rod 1560 is inserted into the bolt hole 1430 in FIG. 14 and the small hole 1540 on the cover, so that the cover and the adjacent electrode holding piece are connected. A pressing piece 1530 with a hole 1520 in the middle on one end of the cover is connected on the sliding rod 1310 in FIG. 13. The pressing piece 1530 compress the spring when stretching the watchband. 1550 is a pipe to hide the wires in FIG. 16.

FIG. 16 shows a detailed internal structure of an electrode holding piece 1230 from FIG. 12. On the electrode holding piece, the electrodes have been mounted on with screws 1610 and 1660. 1660 is used to mount the measuring electrode, and 1610 is used to mount the reference electrode. 1620 is a measuring electrode bus formed by individual wires from each measuring electrode that connected the electrodes to the integrated circuit. The number of wires in the measuring electrode bus equal to the number of measuring electrodes. 1640 is a reference electrode wire connecting the reference electrodes to the integrated circuit. All the reference electrodes are connected to the reference electrode wire 1640 through corresponding wires 1650. The measuring electrode bus and the reference electrode wire are curved, redundant or stretchable, so that they will not be pulled broken when the watchband is stretched.

FIG. 17 shows a schematic diagram of the watch when the watchband is stretched longer. 1550 is the pipe that is exposed after the cover is pulled away from the electrode holding piece.

Various embodiments of the present disclosure have been described above, which are illustrative, not exhaustive, and are not limited to the disclosed embodiments. Without departing from the scope and spirit of the described embodiments, many modifications and variations will be apparent to those of ordinary skill in the art without departing from the scope and spirit of the described embodiments. Therefore, the protection scope of the present disclosure should be subject to the protection scope of the claims.

1. A wearable measuring device to measure skin impedance, which is worn onto a wrist or an ankle, comprising:
one or more stretchable parts, constituting part of a watchband of the measuring device, to allow a length of the watchband to be stretched by 1.3 times or more than 1.3 times of an original length;
an electrode holding part, including six or more individual pieces that are connected with the stretchable parts, wherein, each piece may hold a pair of electrodes;
six or more pairs of electrodes, wherein, one of each pair of the electrodes is a measuring electrode, and the other is a reference electrode, and the six or more pairs of electrodes are arranged on the electrode holding part around the wrist or the ankle;
an integrated circuit part for measuring skin impedance;
and a case, to house the integrated circuit part.

2. The measuring device according to claim 1, wherein, each electrode is made of two pieces of metal sleeved together; the two pieces of metal partially overlap with each other, with a spring connected within to allow the two pieces of metal to move relative to each other;
one piece of metal is in contact with the skin, and the other piece of metal is connected with the integrated circuit through a wire.

3. The measuring device according to claim 1, wherein, all reference electrodes are connected together, and then connect to the integrated circuit; and each measuring electrode is connected to the integrated circuit through an individual wire.

4. The measuring device according to claim 1, wherein, the device is worn onto a wrist or an ankle by passing around a hand or a foot when the stretchable parts are stretched or compressed.

5. The measuring device according to claim 1, wherein, each piece of the electrode holding part has two protruding hollow cylinders along a longitudinal direction of the wrist or the ankle.

6. The measuring device according to claim 5, wherein, in a hollow portion of the cylinder, a diameter on one end close to a skin is greater than a diameter on the other end that is away from the skin.

7. The measuring device according to claim 6, the electrode is inserted into the hollow cylinder from a skin-contacting side of the electrode holding piece; one end of the electrode is in contact with the skin, and the other end of the electrode is mounted onto the electrode holding piece and connected to the integrated circuit through a wire.

8. The measuring device according to claim 1, wherein, the stretchable part is made of silicone, rubber, or plastic.

9. The measuring device according to claim 8, wherein, the stretchable part is punched with pairs of holes along a circumferential direction of the wrist or ankle.

10. The measuring device according to claim 8, wherein, zigzag grooves are cut on the stretchable part; and the wires are connected with the integrated circuit along the groove on the stretchable part.

11. The measuring device according to claim 9, wherein, each pair of holes on the stretchable part are set onto the two cylinders of each electrode holding piece.

12. The measuring device according to claim 11, wherein, each electrode holding piece and the connected stretchable part are covered with a plate, to hide the stretchable part, electrodes and wires.

13. The measuring device according to claim 12, wherein, the adjacent plates are partly overlapped; when the watchband is stretched, the overlapped parts are extended to cover the stretchable part and the wires.

14. The measuring device according to claim 1, wherein, the stretchable parts are springs.

15. The measuring device according to claim 14, wherein, a sliding rod is fixed on each electrode holding piece along a circumferential direction of the wrist or the ankle; and the spring is set on the sliding rod.

16. The measuring device according to claim 14, wherein, the sliding rod is further connected with one end of a cover, next to the spring, and the other end of the cover is connected to an adjacent electrode holding piece; when two electrode holding pieces are pulled away from each other, the end of the cover that is next to the spring slides on the sliding rode and compresses the spring along the sliding rod.

17. The measuring device according to claim 16, wherein, the wires connecting electrodes and the integrated circuits, the springs, the cylinders, the sliding rod are hidden by the covers.

18. The measuring device according to claim 17, wherein, the adjacent covers are partly overlapped; when the watchband is stretched, the overlapped parts are extended to cover the wires, springs, cylinders and sliding rods.
